(19) United States
(12) Patent Application Publication
(10) Pub. No.: US 2026/0345009 A1
(43) Pub. Date: Nov. 5, 2026

(54) Medical imaging device and method for medical imaging

(71) Applicant: KARL STORZ SE & Co. KG, Tuttlingen (DE)

(72) Inventors: Werner Göbel, Überlingen (DE); Lukas Buschle, Mühlheim (DE); Benedikt Köhler, Dürbheim (DE)

(73) Assignee: KARL STORZ SE & Co. KG, Tuttlingen (DE)

(21) Appl. No.: 19/118,542

(22) PCT Filed: Oct. 4, 2023
(86) PCT No.: PCT/EP2023/077373

§ 371 Date: Apr. 4, 2025

(30) Foreign Application Priority Data
Oct. 6, 2022 (DE) 10 2022 125 852.1

(51) Int. Cl. A61B 5/00

(52) U.S. Cl. CPC A61B 5/0071; A61B 5/7425

(57) ABSTRACT

A medical imaging device with an illumination unit configured to illuminate an object and an image capture unit and an observation filter is presented. The observation filter blocks light in a first spectral range including visible light and transmits light in a different, second spectral range. The image capture unit records, through the observation filter, fluorescence images of the object. Illumination light, with a spectrum partly within the first spectral range, is used as excitation light, and light which is emitted from the object and of the spectrum which lies at least partly within the second spectral range is detected. The image capture unit records anatomy images of the object while illumination light, the spectrum of which lies partly within the second spectral range, is used, and light which is remitted from the object and of the spectrum which lies at least partly within the second spectral range is detected.

The invention relates to a medical imaging device, in particular an endoscope device, exoscope device and/or microscope device, a method for medical imaging, associated program code and an associated computer program product.

Imaging devices for performing fluorescence imaging are known from the prior art and can record both fluorescence images and white light images. Suitable excitation light is used to specifically excite fluorescent dyes or, where appropriate, natively occurring fluorescent substances and to detect emitted light and use it for imaging. In order to be able to show anatomical structures in the image region to a user at the same time, a white light image is often recorded in parallel or sequentially. The white light image allows the user to judge whether the anatomical structure is depicted. Fluorescence images and white light images can also be superimposed, allowing anatomical information and fluorescence information to be perceptible and analyzable by a user at the same time.

In addition, imaging devices, such as endoscopic or exoscopic devices that produce multispectral or hyperspectral images, are known from the prior art. Multispectral or hyperspectral images have a spectral dimension in addition to two spatial dimensions, such as a conventional image from a camera. The spectral dimension includes multiple spectral bands (wavelength bands). Multispectral and hyperspectral images differ substantially in the number and width of their spectral bands. Such systems can in principle also be suitable for carrying out fluorescence recordings.

Some imaging devices are known for producing such multispectral or hyperspectral images, especially in the context of medical applications. For example, DE 20 2014 010 558 U1 describes a device for acquiring a hyperspectral image of an examination region of a body. The device includes an input lens for generating an image in an image plane and a slit-shaped aperture in the image plane for masking out a slit-shaped region of the image. The light passing through the aperture is spread out by a dispersive element and recorded by a camera sensor. This allows the camera sensor to record a plurality of spectra, each with an associated spatial coordinate, along the longitudinal direction of the slit-shaped aperture. The device described is further configured to record further spectra along the longitudinal direction of the slit-shaped aperture in a direction different from the longitudinal direction of the slit-shaped aperture. The method underlying this disclosure for generating multispectral or hyperspectral images is also known as the so-called pushbroom method.

In addition to the pushbroom method, there are other methods for generating multispectral or hyperspectral images. In the so-called whiskbroom method, the region under study or else the object is scanned point by point and a spectrum is obtained for each point. In contrast, the staring method involves taking multiple images with the same spatial coordinates. Different spectral filters and/or illumination sources are used from image to image to resolve spectral information. Furthermore, there are methods according to which a two-dimensional multi-color image is broken down into a plurality of individual spectral images using suitable optical elements, such as optical slicers, lenses and prisms, which images are simultaneously acquired on different detectors or detector regions. This is sometimes referred to as the snapshot approach.

As described in DE 10 2020 105 458 A1, multispectral and hyperspectral imaging devices are particularly suitable as endoscopic imaging devices. In this context, multispectral and/or hyperspectral imaging is a fundamental field of application, for example for diagnostics and for assessing the success or quality of an intervention.

Multimodal imaging devices allow the capture of, optionally, white light images and/or multispectral images and/or fluorescence images and/or hyperspectral images. Examples of such imaging devices are multimodal endoscopes and multimodal exoscopes.

Regardless of the exact design, tissue is illuminated in a specific wavelength range to record fluorescence images in order to specifically stimulate fluorescent dye molecules introduced into specific entities, such as tissue regions. The subsequently emitted light with a red-shifted wavelength can be observed through a suitably selected filter, by means of which the excitation light can be blocked. If the wavelength ranges of the excitation light and the emitted light relative to white light used for parallel or sequential white light imaging are in the longer wavelength range, in particular in the near-infrared range, i.e., outside of visible white light, white light imaging is possible through the filter despite these observation filters. In recent times, however, dyes that can be excited with visible light have been increasingly used. The known method of imaging is not feasible here because the filters required for fluorescence imaging impair the white light image by blocking visible light in specific wavelength ranges or usually only or at least primarily transmitting light below a specific wavelength. If fluorescent dyes are used that are to be excited in the visible range, the aforementioned parallel or sequential white light recording is therefore not feasible. However, if the white light image is omitted, it is difficult for the user to correctly assess anatomical structures.

Proceeding from the prior art, the invention is based on the object of allowing fluorescence imaging in a wide range of applications.

This object is achieved according to the invention by an imaging device, a method for medical imaging, program code and a computer program product as described herein and defined in the claims.

According to the invention, a medical imaging device, in particular an endoscope device, exoscope device and/or microscope device, comprises an illumination unit having at least one light source that is configured to provide illumination light to illuminate an object to be imaged. Furthermore, the medical imaging device comprises an image capture unit that comprises at least one image capture sensor and at least one observation filter. The observation filter is configured to block light in a first spectral range, which comprises visible light, and to transmit light in a second spectral range, which differs from the first spectral range. The image capture unit is also configured to record, through the observation filter, fluorescence images of the object to be imaged, during the recording of which images, illumination light, the spectrum of which lies at least partly within the first spectral range, is used as excitation light and, during the recording of which images, furthermore light, which is emitted from the object to be imaged and the spectrum of which lies at least partly within the second spectral range, is detected by the image capture sensor system. In addition, the image capture unit is configured to record anatomy images of the object to be imaged, during the recording of which images, illumination light, the spectrum of which lies at least partly within the second spectral range, is used and, during the recording of which images, furthermore light, which is remitted from the object to be imaged and the spectrum of which lies at least partly within the second spectral range, is detected by the image capture sensor system. Furthermore, the medical imaging device comprises a display generation unit which is configured to generate at least one display from the fluorescence images and the anatomy images.

A method according to the invention can be carried out in particular with the medical imaging device according to the invention. The method comprises providing illumination light for illuminating an object to be imaged. The method further comprises recording fluorescence images of the object to be imaged through an observation filter which is configured to block light in a first spectral range that comprises visible light, and to transmit light in a second spectral range, which differs from the first spectral range, wherein, during the recording of the fluorescence images, illumination light, the spectrum of which lies at least partly within the first spectral range, is used as excitation light, and light, which is emitted from the object to be imaged, the spectrum of which lies at least partly within the second spectral range, is detected. The method also comprises recording anatomy images of the object to be imaged, wherein, during the recording of the anatomy images, illumination light, the spectrum of which lies at least partly within the second spectral range, is used and light which is remitted from the object to be imaged, the spectrum of which lies at least partly within the second spectral range, is detected.

The features according to the invention allow imaging to be carried out in a wide range of applications. In particular, fluorescent dyes that absorb in the visible range can be used, and at the same time a meaningful anatomical image can be provided to a user. This allows different fluorescent dyes to be used equally without having to accept any limitations in the creation of an anatomical image.

Due to the features according to the invention, a fluorescence image and an anatomy image can be recorded even with fluorescence excitation in the visible range, without filters having to be swung in and out of the beam path between the individual recordings. In addition, anatomy images do not have to be completely dispensed with, even if fluorescence is to be excited in the visible range. In principle, it would also be conceivable to irradiate white light and fluorescence excitation light in parallel and to separate fluorescence and white light via the Bayer pattern of a detector. However, due to the associated and expected sensor crosstalk, this requires considerable effort in evaluation and the image quality to be expected may be low.

The imaging device can be a microscopic, macroscopic and/or exoscopic imaging device. The imaging device can be configured as a microscope, macroscope and/or exoscope and/or comprise such. In some embodiments, the imaging device can be an endoscopic imaging device. The imaging device can be an endoscope device. It can comprise an endoscope and/or an endoscope system and/or be configured as such and/or form at least a part and preferably at least a major part and/or main component of an endoscope and/or an endoscope system. “At least a major part” can mean at least 55%, preferably at least 65%, more preferably at least 75%, particularly preferably at least 85% and most preferably at least 95%, in particular with reference to a volume and/or a mass of an object.

In some embodiments, the imaging device is configured to be insertable into a cavity for inspection and/or observation, for example into an artificial and/or natural cavity, such as into the interior of a body, into a body organ, into tissue or the like. The imaging device can also be configured to be insertable into a housing, casing, shaft, tube or other, in particular artificial, structure for inspection and/or observation.

In particular if the imaging device is an exoscopic imaging device, it may be configured to acquire tissue parameters, images of wounds, images of body parts, etc. For example, the imaging device may be configured to image a surgical field.

The image capture sensor technology can be configured to detect light in both the visible and near-infrared range. A minimum detectable wavelength may be at most 500 nm, at most 450 nm, or even at most 400 nm in some embodiments. A maximum detectable wavelength may be at least 800 nm, at least 900 nm, or even at least 1000 nm in some embodiments. The image capture sensor system can, for example, comprise at least one white light image sensor and at least one near-infrared image sensor. In some embodiments, the imaging device comprises a white light camera and/or sensor system for white light imaging. The imaging device may be configured for white light imaging. The anatomy images can be recorded using the white light camera and/or the sensor system for white light imaging.

The image capture unit may include a filter unit with optical observation filters. The filter unit can define several fluorescence modes defined by different observation filters. For example, different edge filters can be used which absorb/block the respectively used spectrum of the corresponding luminous element used for excitation and at least substantially only transmit fluorescent light. The observation filter, which blocks light in the first spectral range, is then part of the filter unit. In some embodiments, the observation filters may also be switchable between a multispectral mode and a fluorescence mode.

The imaging device and in particular an optical system and/or the image capture sensor system may be configured for multispectral and/or hyperspectral imaging, in particular for acquiring and/or generating multispectral and/or hyperspectral image data. Multispectral imaging or multispectral image data can refer in particular to such imaging in which at least two, in particular at least three, and in some cases at least five spectral bands can be acquired and/or are acquired independently of one another. Hyperspectral imaging or hyperspectral image data can refer in particular to imaging in which at least 20, at least 50 or even at least 100 spectral bands can be acquired and/or are acquired independently of one another. The imaging device may operate according to the pushbroom method, and/or the whiskbroom method, and/or according to the staring method, and/or according to a snapshot principle.

For some applications it can be advantageous to be able to use a high spectral resolution. Hyperspectral imaging is then recommended. This can be combined with white light imaging. This makes real-time observation possible via a white light image, even if the capture of spectrally resolved image data only occurs substantially in real time, i.e., for example, several seconds are needed to create a spectrally resolved image. For some applications it can be advantageous to generate spectral image data in real time. This includes, for example, the generation of a spectrally resolved image in less than a second or even multiple times per second. It can be useful to use multispectral imaging in this case. An optionally lower spectral resolution is then offset by a higher refresh rate. Depending on the application, it can be sufficient to consider only a few different spectral ranges and/or wavelengths, for example two or three or four or generally less than ten. In this case, additional white light imaging can optionally be omitted. Spectrally resolved image data that are acquired in real time or deliver several images per second can also be used for monitoring purposes, wherein it is not absolutely necessary to create a reproducible image for a user, but rather the image data can also be processed in the background.

The medical imaging device can have at least a proximal portion, a distal portion and/or an intermediate portion. The distal portion is in particular configured to be introduced into and/or located in a cavity to be examined in an operating state, for example during the diagnostic and/or therapeutic activity. The proximal portion is in particular configured to be arranged outside the cavity to be examined in an operating state, for example during the diagnostic and/or therapeutic activity. “Distal” should be understood to mean, in particular, facing a patient and/or facing away from a user during use. “Proximal” should be understood to mean, in particular, facing away from a patient and/or facing a user during use. In particular, proximal is the opposite of distal. The medical imaging device in particular has at least one, preferably flexible, shaft. The shaft can be an elongated object. Furthermore, the shaft can at least partially and preferably, at least to a large extent, form the distal portion. An “elongated object” is to be understood in particular as an object whose main extension is at least a factor of five, preferably at least a factor of ten and particularly preferably at least a factor of twenty larger than a largest extension of the object perpendicular to its main extension, i.e. in particular a diameter of the object. A “main extension” of an object should be understood in particular as its longest extension along its main extension direction. A “main extension direction” of a component is to be understood in particular as a direction which runs parallel to a longest edge of a smallest imaginary cuboid which only just completely encloses the component.

The image capture unit can be arranged at least partially and preferably at least to a large extent in the region of the proximal portion and/or can form it. In other embodiments, the image capture unit may be arranged at least partially and preferably at least to a large extent in the distal portion and/or can form it. Furthermore, the image capture unit can be arranged at least partially distributed over the proximal portion and the distal portion. The image capture sensor system comprises in particular at least one image sensor. Furthermore, the image capture sensor system can also have at least two and preferably several image sensors, which can be arranged one behind the other. Furthermore, the two and preferably several image capture sensors can have spectral capture sensitivities different from one another so that, for example, a first sensor in a red spectral range, a second sensor in a blue spectral range and a third sensor in a green spectral range is particularly sensitive or comparatively more sensitive than the other sensors. The image sensor can be configured as a CCD sensor and/or a CMOS sensor.

An optical system of the image capture unit can comprise suitable optical elements, such as lenses, mirrors, gratings, prisms, optical fibers, etc. The optical system may be configured to guide object light coming from an imaged object to the image capture sensor system, for example to focus and/or project it.

The image capture unit is in particular configured to generate at least two-dimensional spatial image data. The image capture unit can be spatially resolving in such a way that it provides a resolution of at least 100 pixels, preferably of at least 200 pixels, preferably of at least 300 pixels and advantageously of at least 400 pixels in at least two different spatial directions. The image data are preferably at least three-dimensional, wherein at least two dimensions are spatial dimensions and/or wherein at least one dimension is a spectral dimension. From the image data, multiple spatially resolved images of the image region can be obtained, each of which is assigned to different spectral bands. The spatial and spectral information of the image data can be such that an associated spectrum can be obtained for a plurality of spatial pixels.

In some embodiments, the image capture unit is configured to generate continuously updated image data. The image capture unit can, for example, be configured to generate the image data substantially in real time, which can comprise, for example, generating updated image data at least every 30 seconds, in some cases at least every 20 seconds, and in some cases even at least every 10 seconds or at least every 5 seconds. Preferably, the image capture unit is configured to generate at least the anatomy images and the fluorescence images as well as the display based thereon in real time, for example with a frame rate of at least 5 fps, at least 10 fps, at least 20 fps or even at least 30 fps.

The illumination unit can be multimodal and can comprise a plurality of luminous elements which are optionally activatable independently of one another and which are configured to emit light according to different emission spectra in order to provide the illumination light.

The term “visible light” may refer herein in particular to light with a wavelength between 400 nm and 750 nm. In general, this refers to light that is perceptible to the human eye. In particular, the first spectral range comprises only a sub-range of visible light. The second spectral range can also comprise visible light. The observation filter can be an edge filter, in particular a high-pass filter. The edge of the observation filter or, more generally, the first spectral range can be selected to match the fluorescence dye used. If, for example, fluorescein or Cy5 is used as the dye, excitation occurs in the middle of the visible range, in particular at a distance from its edges, for example at approximately 430 nm for fluorescein or at approximately 660 nm for Cy5. In order to avoid distorting fluorescence images due to excitation light, the excitation light must be blocked accordingly. The first spectral range must then be selected in such a way that the excitation light is blocked. In the case of fluorescein, for example, an edge filter with an edge at approximately 450 nm could be selected. In the case of Cy5, for example, an edge filter with an edge at approximately 680 nm could be selected. The first spectral range and the second spectral range preferably border on one another at least substantially. A spacing between the spectral ranges can, for example, be defined only by a width of the filter edge. There may be a third spectral range which lies between the first spectral range and the second spectral range and in which the observation filter and in particular the transmittance of the observation filter changes significantly depending on the wavelength. As mentioned, this could be the filter edge. The first spectral range and/or the second spectral range may comprise a plurality of sub-ranges which may be spaced apart from one another.

The second spectral range extends, in some embodiments, into the near-infrared range beyond the visible range. In particular, the light detected when recording the anatomy images can be at least in the near-infrared range, for example in a wavelength range beyond 800 nm, beyond 850 nm, or even beyond 900 nm.

The fluorescence images and the anatomy images can be recorded in parallel or sequentially. Preferably, both the fluorescence images and the anatomy images are moving images.

Images can be recorded particularly easily and at short intervals if the image capture unit is configured to record the anatomy images of the object to be imaged through the observation filter. The observation filter can therefore be permanently located in the beam path for both the anatomy recordings and the fluorescence recordings during the recording and display of a superimposed anatomy image and fluorescence image. For example, it can be changed if a different excitation wavelength and/or a different imaging mode is selected. The observation filter is located in particular in front of an optical input of an image capture sensor of the imaging device. As mentioned, this can comprise sensors that respond in different spectral ranges; the observation filter is nevertheless expediently arranged in front of all of these sensors, i.e., in front of the image capture sensors. This allows different filters to be used easily without having to integrate the filters directly into the sensor system.

In other embodiments, the observation filter can be integrated into the image capture sensor. In particular for multi-chip image sensors, different image sensors can be equipped with different observation filters. The capture of the anatomy images and the capture of the fluorescence images can then be carried out using disjoint groups of image sensors. Images can be recorded in parallel. In this case, the observation spectral range used for fluorescence image capture is not available for anatomy image capture.

A smallest wavelength of the first spectral range can be greater than 430 nm and in particular greater than 500 nm. A longest wavelength of the first spectral range can be less than 800 nm and in particular less than 700 nm. In some embodiments, the illumination light used as excitation light for fluorescence imaging is narrowband. For example, the illumination light can come from at least one colored LED, laser diode or other at least substantially monochromatically emitting luminous element.

In some embodiments, the medical imaging device comprises an illumination device that includes the illumination unit. The illumination device may comprise an optical interface for optically connecting an imaging device. The illumination unit can be configured to supply illumination light to the optical interface. The illumination unit can be multimodal and can comprise a plurality of luminous elements which are optionally activatable independently of one another and which are configured to emit light according to different emission spectra in order to provide the illumination light. The illumination unit may be operable in at least one multispectral mode in which a first group of the luminous elements is at least temporarily activated and in which the illumination unit provides illumination light for multispectral imaging. The illumination unit may further be operable in at least one fluorescence mode in which a second group of the luminous elements is at least temporarily activated and in which the illumination unit provides illumination light for fluorescence imaging. The luminous elements can comprise at least one luminous element which is included in both the first group and the second group.

Moreover, a method for generating illumination light for an imaging unit by means of an illumination device can be provided. The illumination device comprises an optical interface for the optical connection of an imaging unit, and an illumination unit that is designed to provide illumination light to the optical interface, wherein the illumination unit comprises a plurality of luminous elements which are optionally activatable independently of one another and which are configured to emit light according to different emission spectra in order to provide the illumination light. The method comprises the step of at least temporarily activating a first group of the luminous elements to provide illumination light for multispectral imaging and the step of at least temporarily activating a second group of the luminous elements to provide illumination light for fluorescence imaging. At least one of the luminous elements is activated at least temporarily both when the first group of luminous elements is activated at least temporarily and when the second group of luminous elements is activated at least temporarily.

The optical interface can be either detachable or connectable. In addition, the optical interface can be combined with a mechanical interface so that an optical connection is automatically established, for example, when the imaging unit is mechanically coupled.

The luminous elements can comprise single-color LEDs (light-emitting diodes) and/or laser diodes. Furthermore, at least one of the luminous elements can be a white light LED or other white light source. In some embodiments, the illumination unit comprises at least one blue luminous element, at least one red luminous element, at least one dark red luminous element and at least one near-IR luminous element (near-infrared luminous element), in particular LEDs or laser diodes in each case. In addition, the illumination unit can comprise at least one white light LED or other white light source.

The first group can comprise at least two luminous elements that emit spectrally differently. A high degree of efficiency in multispectral imaging can be achieved if the multispectral mode comprises different states in which a specific luminous element or a specific type of luminous element is activated at least temporarily. This allows targeted illumination in a specific spectral range, allowing different spectral images to be captured. Different luminous elements activated in different states can serve as different support points for multispectral imaging. At least one of these support points can be selected such that it is adapted to characteristic points of absorption spectra of physiologically relevant components, for example to an isosbestic point of the hemoglobin oxygenation curve. Multispectral imaging can additionally include the use of appropriate observation filters.

Furthermore, the second group can comprise at least two luminous elements that emit spectrally differently. The fluorescence mode can comprise different submodes and/or states in which a specific luminous element or a specific luminous element type is activated at least temporarily. This allows targeted excitation in a certain spectral range, so that fluorescence imaging can be carried out, for example, for a specifically selected dye. In other words, the at least one luminous element contained in both the first group and the second group can be used for both the multispectral mode and the fluorescence mode.

In some embodiments, the first group comprises only some but not all of the luminous elements. Alternatively or additionally, in some embodiments, the second group comprises only some but not all of the luminous elements. In the multispectral mode only luminous elements of the first group, in particular, are activated at least temporarily, whereas luminous elements that do not belong to the first group are deactivated. In the fluorescence mode only luminous elements of the second group, in particular, are activated at least temporarily, whereas luminous elements that do not belong to the second group are deactivated. In general, it shall be understood that the luminous elements can comprise different luminous element types and that exactly one luminous element of each of the different luminous element types, in particular, can be present. It shall be understood that mixed operating modes can also occur according to the invention, in which said modes are used sequentially. For example, multispectral imaging and fluorescence imaging can be performed sequentially.

Synergy with regard to the use of a luminous element for different modes and associated efficiency gains can be achieved in particular if at least one luminous element contained in both the first group and the second group emits light in the red spectral range, in particular in a spectral range between 600 nm and 680 nm, for example between 610 nm and 650 nm or between 620 and 660 nm or between 630 and 670 nm. The spectral range can be narrowband and cover the 660 nm wavelength. “Narrowband” may include a spectral width of at most 80 nm, in particular at most 40 nm or even at most 20 nm. This at least one luminous element can be configured to excite dyes absorbing in the red spectral range and to contribute to the illumination in the red spectral range for multispectral imaging.

In some embodiments, the illumination unit may be operable in at least one white light mode in which the illumination unit provides illumination light for white light imaging. The illumination light for white light imaging can be broadband white light. Alternatively, the illumination light for white light imaging can comprise a plurality of narrow wavelength bands that are separated from one another, for example a blue, a red and a dark red band. “Dark red” is to be understood as having a “longer wavelength than red” and refers to the spectral position, but not to light intensity. The illumination light for white light imaging can be a mixture of light from different luminous elements.

In the white light mode, a third group of luminous elements may be activated at least temporarily to provide the illumination light for white light imaging. The luminous elements can comprise at least one luminous element which is contained both in the first group and/or second group and in the third group. In some cases, the third group may comprise only some but not all of the luminous elements. In the white light mode only luminous elements of the third group, in particular, are activated at least temporarily, whereas luminous elements that do not belong to the third group are deactivated. In other words, the illumination unit may comprise luminous elements that serve one, two or all three of the aforementioned illumination modes. This allows for a plurality of luminous elements to be used multiple times.

At least one luminous element contained both in the first group and/or second group and in the third group can emit light in the red spectral range, in particular in a spectral range between 600 nm and 680 nm, for example between 610 nm and 650 nm or between 620 and 660 nm or between 630 and 670 nm. The advantages of using luminous elements together are particularly evident when at least one red luminous element can be used for all three modes.

At least one luminous element contained both in the first group and/or second group and in the third group can emit light in the blue spectral range, in particular in a spectral range between 440 and 480 nm. At least one blue luminous element can be conveniently used both in the fluorescence mode and in the white light mode.

Generally speaking, the luminous elements, as mentioned, can comprise at least one, in particular blue, luminous element which emits light in a spectral range between 440 and 480 nm. Moreover, the luminous elements, as mentioned, can comprise at least one, in particular red, luminous element which emits light in a spectral range between 600 and 680 nm, for example between 610 nm and 650 nm or between 620 and 660 nm or between 630 and 670 nm. Alternatively or additionally, the luminous elements can comprise at least one, in particular dark red, luminous element which emits light in a spectral range between 750 and 790 nm. Alternatively or additionally, luminous elements can comprise at least one, in particular near-IR emitting, luminous element that emits light in a spectral range between 920 and 960 nm. In addition, the luminous elements can include a white light luminous element. A compact and versatile illumination unit can be provided in particular if at least one luminous element of each of the above-mentioned luminous element types is present. For example, in fluorescence mode, the blue and the red luminous element can be used, and in the case of suitable dyes, the dark red luminous element may also be used. In multispectral mode, the dark red and near-IR emitting luminous elements can be used. In white light mode, the white light luminous element can be used. In white light mode, it can be supplemented by the blue luminous element and, if necessary, the red luminous element. This makes it possible to supplement wavelength ranges by means of colored luminous elements in which the white luminous element provides a reduced intensity, for example due to its construction but in particular due to filters and optical elements of the illumination unit. In addition, the colored luminous elements can be used to adjust a color temperature for white light imaging.

In some embodiments, the second group comprises a single luminous element and/or a single type of luminous elements. For example, a white light luminous element, a red luminous element and an IR-emitting luminous element can be provided, with particular reference being made to the above values as regards possible spectral ranges. The first group can then, for example, comprise the red and the IR-emitting luminous element. The second group can comprise the IR-emitting luminous element, in particular as the only luminous element or as the only type of luminous element.

A favorable arrangement of luminous elements is made possible in particular if the illumination unit comprises at least one crossed beam splitter, by means of which light can be deflected from opposite input sides to an output side, wherein at least one of the luminous elements is arranged on the opposite input sides of the crossed beam splitter. In some embodiments, two or more crossed beam splitters may be provided that are arranged optically one behind the other. The at least one crossed beam splitter can comprise two beam splitter elements, the transmittance of which is adapted to the respectively associated luminous element. The beam splitter elements each comprise, in particular, a notch filter so that they reflect in a narrow spectral band but otherwise transmit. The spectral position and/or width of the corresponding notch can be adapted to the spectral range of the respectively associated luminous element, so that its light is redirected, but light from other luminous elements is at least largely transmitted.

In some embodiments, the luminous elements can comprise at least four narrowband emitting single-color luminous elements, each with different spectral ranges, and at least one broadband emitting white light luminous element. In this regard, reference is also made to what has been said above in connection with colored luminous elements.

A wide range of functions in combination with a compact design and the utilization of synergy effects when using luminous elements can be achieved in particular if the illumination unit is operable in at least one hyperspectral mode in which a plurality of luminous elements is activated, the emission spectra of which together cover at least a spectral range from 450 nm to 850 nm, and in which the illumination unit provides illumination light for hyperspectral imaging. This may in particular include all of the luminous elements.

It shall be understood that, in particular when using laser diodes, suitable polarization filters can be used for the optical filters mentioned herein. Furthermore, in particular when using laser diodes, at least one crossed beam splitter can be used, the beam splitter elements of which are provided with polarization filters. Selective transmittance can then be achieved by combining different polarizations.

The display generated by the display generation unit from the fluorescence images and the anatomy images may comprise a combination display, in particular an overlay display based on a combination of fluorescence images and anatomy images. A user can then easily assign fluorescence information to anatomical information. In particular, the fluorescence images and the anatomy images can be directed at the same object, in particular the same anatomy. Image portions can differ from one another.

The imaging device may comprise a visual display and/or be configured to be connected to a visual display. An appropriate interface may be available for this purpose. The display generation unit can be configured to generate the displays for the visual display. The display may, for example, comprise a screen on which the display is made available to a user.

In some embodiments, the image capture unit is configured to record the fluorescence images and the anatomy images in real time. Furthermore, the display generation unit can be configured to generate the at least one display in real time. In this way, an anatomical and a fluorescence-based moving image can be made available to a user, even if excitation occurs in the visible range.

As mentioned, in some embodiments, the image capture unit may be configured to acquire the fluorescence images and the anatomy images sequentially. It can then be advantageous if the display generation unit is configured to assign sequentially recorded fluorescence images and anatomy images to one another, in particular in terms of time, and to generate the at least one display based on assigned fluorescence images and anatomy images. The assignment can be carried out frame-synchronously. “Frame-synchronously” is understood to mean that assigned fluorescence images and anatomy images are temporally correlated. This does not necessarily mean simultaneity in the narrow sense. In addition, the term “frame-synchronously” also comprises situations in which a first number of fluorescence images and a different second number of anatomy images are recorded alternately. For example, according to the invention it can be provided that an update rate for anatomy images is greater than an update rate for fluorescence images. The recording is nevertheless frame-synchronous within the meaning of this disclosure if the assignment is made in such a way that there is a temporal connection between the images displayed. In other words, the combination display can be based on at least one fluorescence image and at least one anatomy image that were recorded within a predetermined period of time, for example within a period of at most 1000 ms, at most 500 ms, at most 100 ms or even at most 50 ms.

In addition to the above embodiments on the image capture sensor system, it can generally be provided that the image capture sensor system comprises at least one first image capture sensor which is configured to capture image information for the fluorescence images, and that the image capture sensor system further comprises at least one second image capture sensor which is configured to capture image information for the anatomy images. The image capture sensors can be combined by means of at least one optical element in such a way that detected light always or at least selectively falls on several or all image capture sensors simultaneously. The first image capture sensor may comprise at least one near-infrared image sensor. The second image capture sensor may comprise a white light image capture chip. Alternatively or additionally, the second image capture sensor may comprise different color channels, for example a red, a green and a blue color channel. This can be done, for example, by using a color filter with a suitable pattern. In some embodiments, for example, a Bayer sensor is used, but other arrangements are also possible according to the invention. Furthermore, it can be provided that a separate two-dimensionally resolving image sensor is used for each color channel. It is understood that in some embodiments, a fluorescence image may additionally or alternatively be generated by means of the first image capture sensor and/or that an anatomy image may additionally or alternatively be generated by means of the second image capture sensor. For example, the anatomy images may comprise multiple anatomy images and/or be based on multiple individual images associated with different color channels, such as images from a red color channel, a green color channel, a blue color channel, and/or a near-infrared color channel. Alternatively or additionally, a single anatomy image may comprise multiple color channels, for example a red color channel, a green color channel, a blue color channel and/or a near-infrared color channel.

In other embodiments, the image capture sensor system comprises a single image capture sensor, in particular a single-chip image capture sensor and/or a single-chip camera head that images in both the visible range and the near-infrared range.

In some embodiments, the image capture sensor may be configured to record fluorescence images and anatomy images simultaneously. “Simultaneous” means in particular a temporal simultaneity, which differs, for example, from a sequential recording of fluorescence images and anatomy images. In this case, for example, the recording of an anatomy image and the simultaneous recording of a fluorescence image can be triggered within a period of time of no more than 500 ms, no more than 100 ms or even no more than 20 ms. However, “simultaneous” may comprise that the corresponding images take different amounts of time to record, for example if different exposure times are used.

Available image information can be used in a targeted manner and to generate a meaningful anatomy image, in particular if the display generation unit is configured to generate at least one anatomy image from a plurality of, in particular single-colored, individual images and/or from a plurality of color channels of anatomy images. In this way, the anatomy image can be synthesized from several color channels, which can at least partly compensate for the loss of a specific color channel and/or a specific spectral range due to the observation filter. Depending on the image capture sensor technology used, individual image sensors can also provide individual images, for example single-colored individual images, such as a blue, green, red and/or near-infrared individual image, or also different and possibly partly multi-colored individual images, such as an RGB individual image and a near-infrared individual image.

In some embodiments, the anatomy image corresponds to a white light image with multiple color channels, in which at least one color channel from the visible range is replaced by at least one color channel that lies outside the visible range, in particular in the near-infrared range. This allows an almost natural anatomy image to be created, even if spectral information is lost due to the observation filter.

The invention also relates to program code which, when executed in a processor, is designed to effect implementation of a method according to the invention.

Furthermore, the invention relates to a program code comprising a computer-readable medium on which the program code according to the invention is stored.

The devices and systems according to the invention and the methods according to the invention should not be limited to the application and embodiment described above. In particular, they can have a number of individual elements, components and units as well as method steps, which differ from a number mentioned herein, in order to fulfill a function described herein. In addition, for the ranges of values specified in this disclosure, values within the stated limits shall also be deemed to be disclosed and to be usable in any manner.

It is in particular pointed out that all features and properties described with regard to a device, but also procedures, can be analogously transferred to methods and can be used within the meaning of the invention and are considered to be co-disclosed. The same applies in the opposite direction. This means that structural features mentioned in relation to methods, i.e., features relating to the device, can also be taken into account, claimed and also counted as part of the disclosure within the scope of the device claims.

The present invention is described below by way of example with reference to the accompanying figures. The drawings, the description, and the claims contain numerous features in combination. A person skilled in the art will also, expediently, consider the features individually and use them in combination as appropriate in the context of the claims.

If there is more than one example of a particular object, only one of them may be provided with a reference sign in the figures and in the description. The description of this example can be transferred accordingly to the other examples of the object. If objects are named using numerical words, such as first, second, third object, etc., these are used to name and/or assign objects. Accordingly, for example, a first object and a third object may be included, but not a second object. However, a number and/or sequence of objects could also be derived using numerical words.

In the drawings:

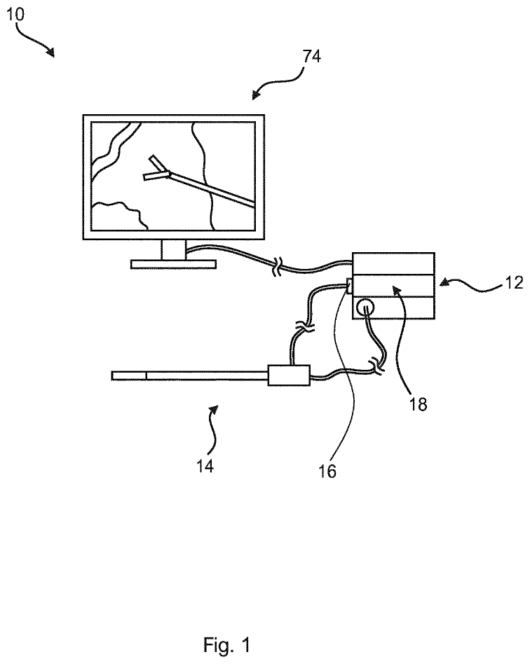

FIG. 1 shows a schematic representation of an imaging device having an illumination device;

FIG. 2 shows a schematic representation of the illumination device;

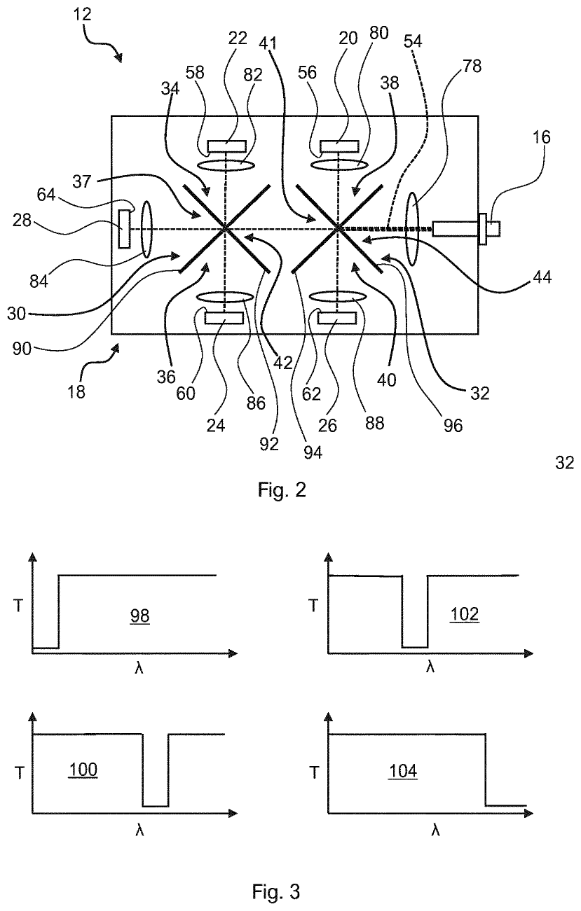

FIG. 3 shows schematic transmission curves of beam splitter elements of the illumination device;

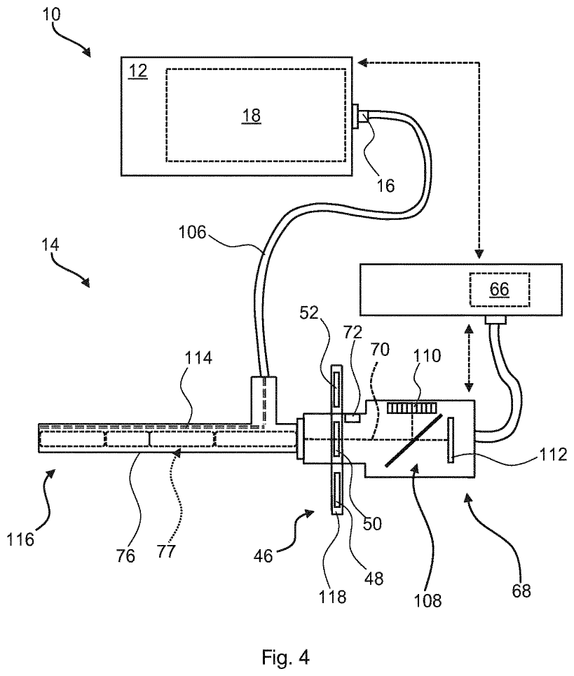

FIG. 4 shows a schematic representation of the imaging device;

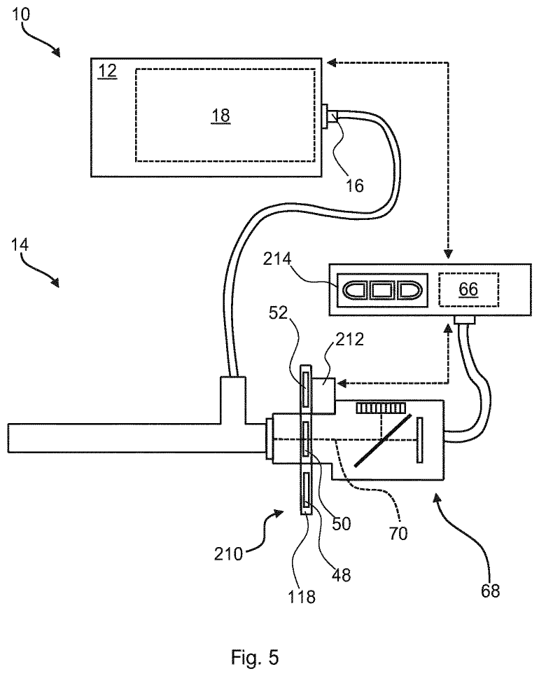

FIG. 5 shows a schematic representation of another embodiment of the imaging device;

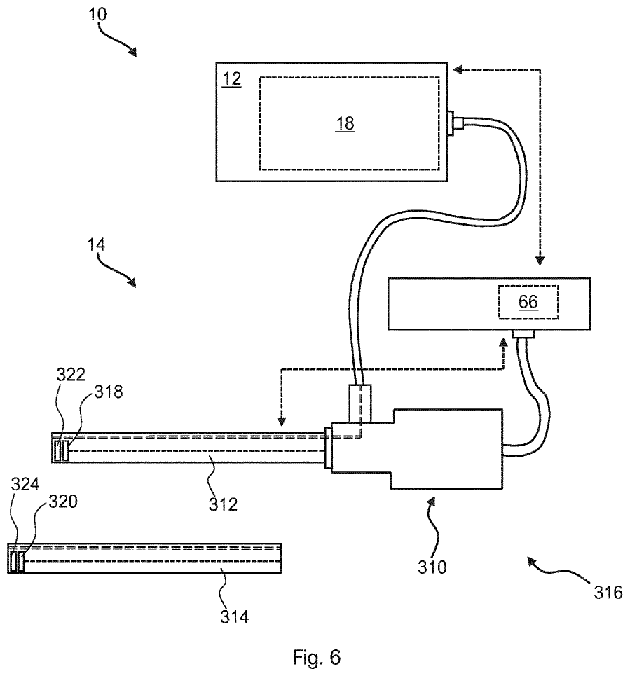

FIG. 6 shows a schematic representation of yet another embodiment of the imaging device;

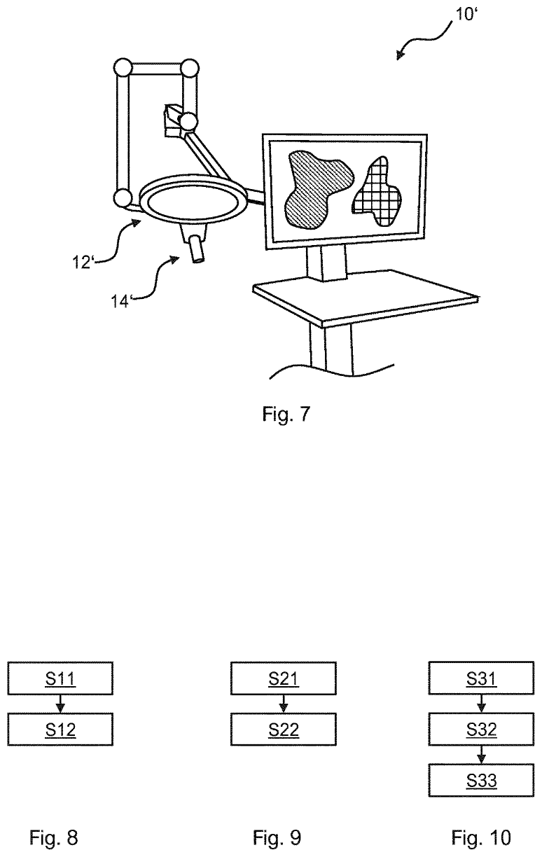

FIG. 7 shows a schematic perspective representation of another embodiment of the imaging device;

FIG. 8 shows a schematic flow chart of a method for generating illumination light for an imaging unit by means of an illumination device;

FIG. 9 shows a schematic flow diagram of a method for operating an imaging device;

FIG. 10 shows a schematic flow diagram of a method for operating an imaging device;

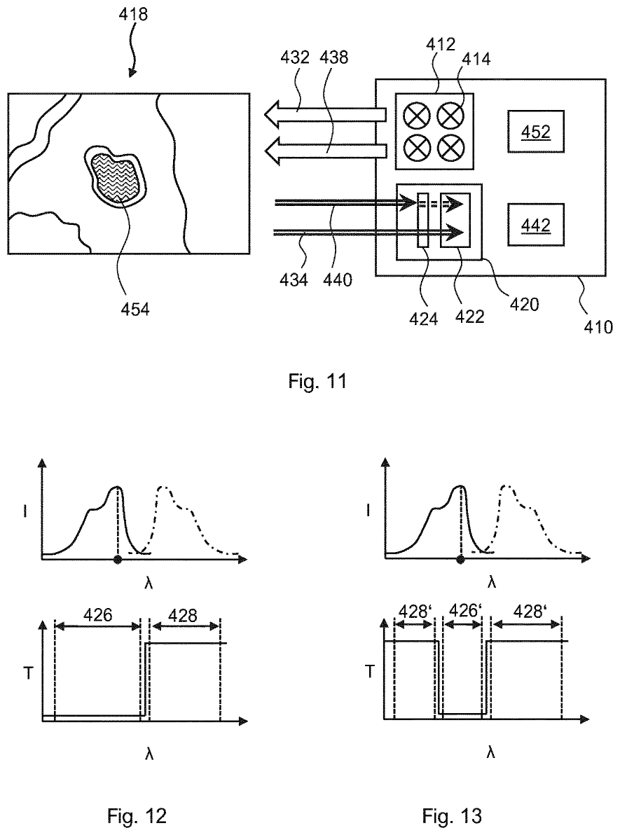

FIG. 11 shows a schematic representation of a medical imaging device;

FIG. 12 shows schematic spectra of a fluorescent dye and a transmission curve of an associated observation filter;

FIG. 13 shows schematic spectra of the fluorescent dye and a transmission curve of an alternative corresponding observation filter;

FIG. 14 shows schematic representations of a series of fluorescence images and anatomy images;

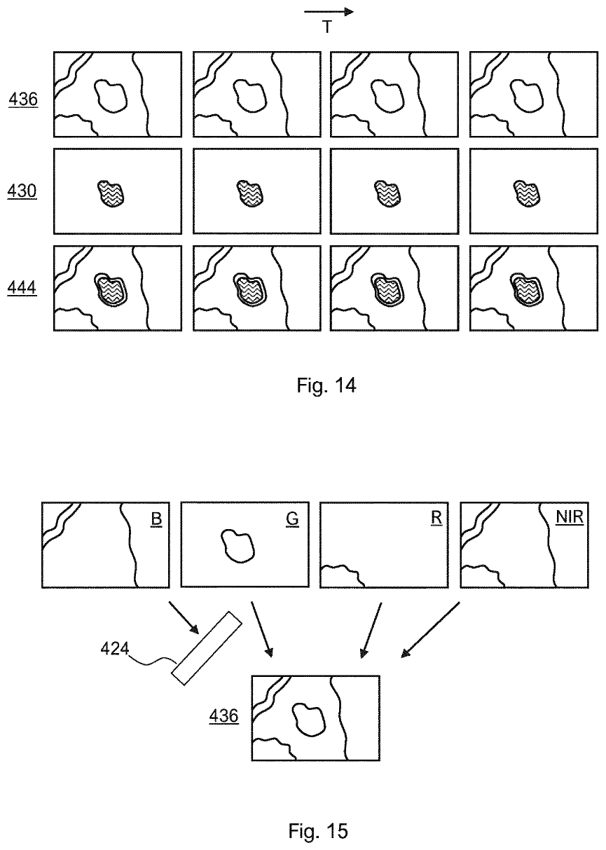

FIG. 15 shows a schematic representation of color channels of an anatomy image;

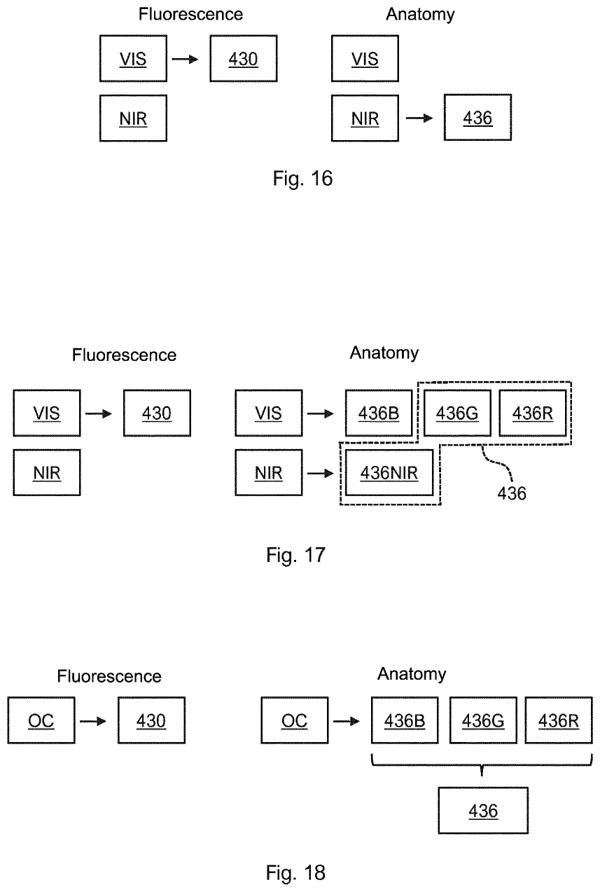

FIG. 16 shows a schematic diagram illustrating a first example of calculating fluorescence images and anatomy images;

FIG. 17 shows a schematic diagram illustrating a second example of calculating fluorescence images and anatomy images;

FIG. 18 shows a schematic diagram illustrating a third example of calculating fluorescence images and anatomy images;

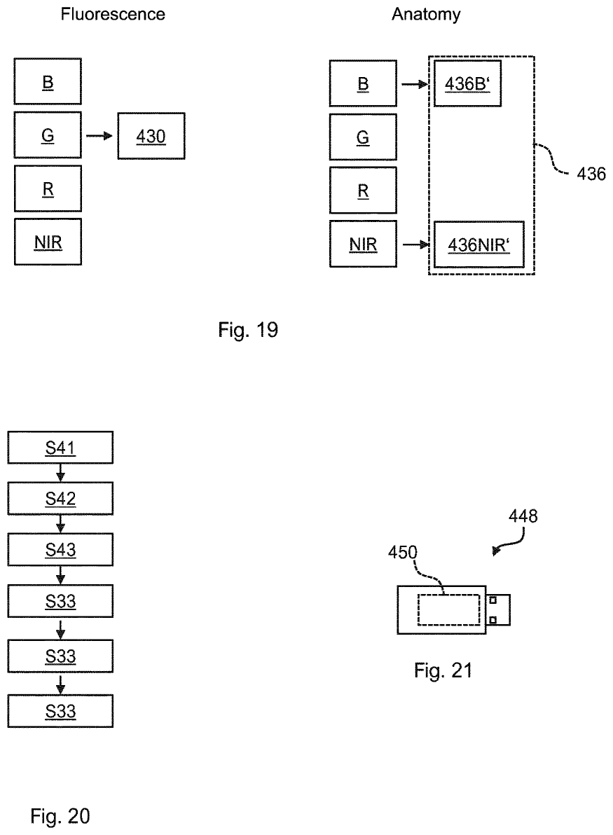

FIG. 19 shows a schematic diagram illustrating a fourth example of calculating fluorescence images and anatomy images;

FIG. 20 shows a schematic flow diagram of a method for medical imaging; and

FIG. 21 shows a schematic display of a computer program product;

FIG. 1 shows a schematic representation of an imaging device 10. In the instance shown by way of example, the imaging device 10 is an endoscopic imaging device, specifically an endoscope device. Alternatively, the imaging device 10 could be an exoscopic, a microscopic or a macroscopic imaging device. The imaging device 10 is shown by way of example as a medical imaging device. The imaging device 10 is provided, for example, for examining a cavity.

The imaging device 10 comprises a medical imaging unit 14. In the shown case, this is an endoscope.

The imaging device 10 further comprises an illumination device 12 having an optical interface 16, and an illumination unit 18. The imaging unit 14 can be optically connected to the optical interface 16. The optical interface 16 can be part of an optical-mechanical interface that can be optionally detachable and connectable. The illumination apparatus 14 can optionally be decoupled from the illumination device 12. The illumination unit 18 is designed to provide illumination light to the optical interface 16. When imaging by means of the imaging unit 14, the illumination unit 18 can accordingly provide the required illumination light, which is guided to the illumination apparatus 14 and from there coupled out onto an object to be imaged, such as a situs.

In the illustrated case, the imaging device 10 further comprises a visual display unit on which images can be displayed that are based on image data acquired by means of the imaging unit 14. These can be video images, still images, overlays of different images, partial images, image sequences, etc.

The imaging device 10 is multimodal. By way of example, the imaging device can be operated in three basic modes: a multispectral mode, a fluorescence mode and a white light mode. It can further be provided for the imaging device 10 to be operable in a hyperspectral mode in addition or as an alternative to the multispectral mode.

The illumination device 12 is multimodal. The illumination device 12 is operable in different illumination modes in which it provides light for different imaging modes. Here, the illumination device 12 is operable in three basic modes, namely a multispectral mode, a fluorescence mode and a white light mode. The imaging unit 14 is also operable in different operating modes, specifically also in at least a multispectral mode, a fluorescence mode and a white light mode. In the corresponding operating mode of the imaging device 10, the modes of the illumination device 12 are matched to one another.

FIG. 2 shows a schematic representation of the illumination device 12. The illumination unit 18 comprises a plurality of independently activatable luminous elements 20, 22, 24, 26, 28. They are configured to emit light according to different emission spectra in order to provide illumination light, i.e., the relevant emission spectrum differs from luminous element to luminous element.

The luminous elements 20, 22, 24, 26, 28 are designed as LEDs, for example. Specifically, a first luminous element 20 is configured as a red LED, a second luminous element 22 as a dark red LED, a third luminous element 24 as a blue LED and a fourth luminous element 26 as a near-IR LED. The colored luminous elements 20, 22, 24, 26 each emit in a narrow band, for example with an emission peak for example at wavelengths 660 nm (first luminous element 20), 770 nm (second luminous element 22), 460 nm (third luminous element 24) and 940 nm (fourth luminous element 26).

A fifth luminous element 28 is further provided, which in the present case is a white light luminous element, for example a white light LED. The fifth luminous element 28 emits, for example, in a spectral range of about 400 to 700 nm. In other embodiments, laser diodes can also be used, in particular as colored luminous elements.

Depending on the illumination mode, some of the luminous elements 20, 22, 24, 26, 28 are activated at least temporarily, whereas other luminous elements 20, 22, 24, 26, 28 may not be used in the illumination mode in question.

In the present case, a first group comprises the first luminous element 20 and the fourth luminous element 26. The first group may additionally comprise luminous element 22 and/or luminous element 24. The first group is used for multispectral imaging, with the contained luminous elements 20, 26 and, if applicable, 22 and 24 each serving as support points. In multispectral mode, for example, the first luminous element 20 is initially used for illumination and an image is taken. Then, the fourth luminous element 26 is used for illumination and an image is taken. The images are based on remission, i.e., the light scattered back from the object being imaged is considered. The two different support points can be used to obtain spectral information about the object to be imaged. For example, certain tissue types, a perfusion state, a tissue condition or the like can be assessed in this way.

Furthermore, a second group comprises the first luminous element 20, the second luminous element 22 and the third luminous element 24. The second group is used for illumination in fluorescence imaging. For example, objects that have been colored with appropriately selected dyes can be viewed. Also, different dyes can be introduced into different types of tissue or the like, which are viewed simultaneously. By specifically exciting a particular dye, it is excited to fluoresce. The fluorescent light is then imaged. The first luminous element 20 is suitable, for example, for exciting the dye cyanine 5.5 (Cy5.5). The second luminous element 22 is suitable for exciting the dye indocyanine green (ICG). The third luminous element 24 is suitable for exciting the dye fluorescein.

Furthermore, a third group comprises the fifth luminous element 28. In the present embodiment, the third group moreover comprises the first luminous element 20 and the third luminous element 24. The third group is used to provide illumination light for white light imaging. For this purpose, white light from the fifth luminous element 28 can be mixed with light from certain colored luminous elements, whereby spectral losses can be compensated and/or a color temperature can be specifically adjusted.

It can be seen that some of the luminous elements 20, 22, 24, 26, 28 are assigned to a plurality of groups, for example the first luminous element 20 to all three groups and the third luminous element 24 and possibly also the second luminous element 22 to the second group and third group.

Alternatively or additionally, it may also be provided for some or all of the luminous elements 20, 22, 24, 26, 28 to be used in a hyperspectral mode. A broad spectrum of excitation is then generated. In combination with a suitable hyperspectral detector, spectral information about the object to be imaged can then be captured across the entire visible and near-IR spectrum. For this purpose, the imaging unit 14 may comprise a pushbroom arrangement as a hyperspectral detector. In other embodiments, a whiskbroom arrangement, a staring arrangement and/or a snapshot arrangement is used. The imaging unit 14 can be a hyperspectral imaging unit. Regarding different methods of hyperspectral imaging and components required for this, reference is made to the article “Review of spectral imaging technology in biomedical engineering: achievements and challenges” by Quingli Li et al., published in Journal of Biomedical Optics 18(10), 100901, October 2013, and to the article “Medical hyperspectral imaging: a review” by Guolan Lu and Baowei Fei, published in Journal of Biomedical Optics 19(1), 010901, January 2014.

The illumination unit 18 comprises two crossed beam splitters 30, 32. They each comprise an output side 42, 44, an input side 37, 41 opposite the output side 42, 44 and two input sides 34, 36, 38, 40 opposite one another. All input sides 34, 36, 37, 38, 40, 41 lead incident light to the corresponding output side 42, 44. The output side 42 of a first crossed beam splitter 30 faces an input side 41 of the second crossed beam splitter 32. The output side 44 of the second crossed beam splitter 32 faces the optical interface 16. The two crossed beam splitters 30, 32 are preferably arranged coaxially to one another and/or to the optical interface.

The illumination unit 18 can comprise suitable optical elements, such as lenses and/or mirrors (not shown). A plurality of lenses 78, 80, 82, 84, 86, 88 is shown by way of example in FIG. 2. A lens 78 is assigned, for example, to the optical interface 16 and couples light coming from the output side 44 of the second crossed beam splitter 32 into the optical interface 16. Furthermore, each of the luminous elements 20, 22, 24, 26, 28 can be assigned a lens 80, 82, 84, 86, 88. A particularly high degree of compactness can be achieved in particular if the luminous elements 20, 22, 24, 26, 28 are each arranged without an intermediate mirror on input sides 34, 36, 37, 38, 40 of the at least one crossed beam splitter 30, 32. The luminous elements 20, 22, 24, 26, 28 can then be moved very close to the at least one crossed beam splitter 30, 32.

The crossed beam splitters 30, 32 each comprise two beam splitter elements 90, 92, 94, 96. They can basically be partially transmitting, so that light from all input sides 34, 36, 37, 38, 40, 41 is redirected to the relevant output side 42, 44. In the present embodiment, the beam splitter elements 90, 92, 94, 96 are selectively light-transmissive. This is illustrated with further reference to FIG. 3. The beam splitter elements 90, 92, 94, 96 can be filters that reflect only in a defined region, but otherwise have high transmission. FIG. 3 shows transmission curves 98, 100, 102, 104 of the beam splitter elements 90, 92, 94, 96 of the two crossed beam splitters 30, 32. Each of the colored luminous elements 20, 22, 24, 26 or each of the opposite input sides 34, 36, 38, 40 is assigned one of the beam splitter elements 90, 92, 94, 96. The beam splitter elements 90, 92, 94, 96 are selected such as to each reflect in the wavelength range in which the associated luminous element 20, 22, 24, 26 emits, but to otherwise largely transmit. For this purpose, notch filters can be used in the medium wavelength range, which can have, for example, transmission spectra 100 and 102. At spectral edges, high-pass or low-pass filters can be used instead of notch filters (see transmission spectra 98 and 104).

Due to the specific transmission spectra 98, 100, 102, 104 of the crossed beam splitters 30, 32, light from the fifth luminous element 28 is spectrally clipped. It may therefore be expedient, in the manner already mentioned, to supplement the light blocked by the beam splitters 30, 32 in a targeted manner by means of the luminous elements 20 and 24, if necessary also 22 and/or 26. This allows for supplementing specifically in those spectral ranges in which the beam splitters 30, 32 absorb and/or reflect light from the fifth luminous element 28, but in any case do not transmit it to the optical interface 16. The additionally used luminous elements 20, 24 and, if applicable, 22 are preferably operated with reduced power or with adjusted power. The aim here may be to at least largely restore the original spectrum of the fifth luminous element 28.

In some embodiments, the fifth luminous element 28 may alternatively be a green luminous element or, more generally, a colored luminous element that emits primarily in the spectral range that the at least one beam splitter 30, 32 transmits. For example, in such embodiments, the fifth luminous element 26 may be an LED having an emission peak at about 530 nm. A green laser diode can also be used for this purpose. Here, it can be provided that color mixing takes place in white light mode and, in particular, that no individual white light source, such as a white light LED is used, but that white light from separate luminous elements is specifically mixed.

It shall be understood that in case of suitable dyes, such a green luminous element can also be used in fluorescence mode. Alternatively or additionally, it could be used in multispectral mode.

The illumination unit 18 defines a common optical path 54 into which emitted light from the luminous elements 20, 22, 24, 26, 28 can be coupled. The common optical path 54 extends from the output side 44 of the second crossed beam splitter 32 to the optical interface. In the present case, the common optical path 54 is arranged coaxially with the fifth luminous element 26.

In the embodiment shown, the luminous elements 20, 26 of the first group are arranged such that light emitted by the luminous elements 20, 26 travels through a light path of at least substantially the same length from the relevant luminous element 20, 26 to the optical interface 16. The luminous elements 20, 26 of the first group each have a light-emitting surface 56, 58. The light-emitting surfaces 56, 62 are arranged equidistantly with respect to the common optical path 54. In the present case, this is achieved in that the two luminous elements 20, 26 are arranged at the same distance from the beam splitter 32 assigned to them (in the present case, by way of example, the second beam splitter 32), in particular from its opposite input sides 38, 40. The light is coupled by the crossed beam splitter 32 into the common optical path 54.

The beam splitters 30, 32 are in particular arranged such that light-emitting surfaces 56, 58, 60, 62, 64 of the luminous elements 20, 22, 24, 26, 28 are each arranged equidistantly with respect to their associated crossed beam splitter 30, 32.

By using crossed beam splitters 30, 32 and luminous elements 20, 22, 24, 26, 28 that can be used together for different modes, the illumination unit 18 or the illumination device 12 has a high degree of compactness. In addition, the equidistant arrangement ensures that no spectral shifts occur when the imaging unit 14 or its light guide is rotated relative to the optical interface 16.

It shall be understood that a different number of luminous elements 20, 22, 24, 26, 28 and/or a different number of crossed beam splitters 30, 32 can be used. The use of crossed beam splitters 30, 32 has proven to be particularly useful. In other embodiments, however, other types of beam splitters and/or other optical elements can be used to couple light from the luminous elements 20, 22, 24, 26, 28 into the optical interface 16.

FIG. 4 shows a schematic representation of the imaging device 10. The imaging unit 14 is optically coupled to the optical interface 16, for example via a light guide 106, such as at least one optical fiber.

The imaging device 10 has a controller 66 which is configured to automatically match an operating state of the imaging unit 14 and an illumination mode of the illumination unit 18 to one another. In the present case, a user can specify the operating mode of the imaging unit 14 by means of a user action. The controller 66 then adjusts the appropriate illumination mode of the illumination unit 18. Alternatively or additionally, the user can adjust a specific illumination mode of the illumination unit 18 by means of a user action. The controller 66 can then adjust an appropriate operating mode of the imaging unit 14. The illumination device 12 and/or the imaging device 10 has, for example, a user interface via which the user can enter corresponding commands.

The imaging unit 14 comprises a camera unit 68 and a distal shaft 76. The distal shaft 76 is optically coupled to the camera unit 68. The camera unit 68 can have a connection for the distal shaft 76, wherein the distal shaft 76 can be optionally decouplable and couplable.

The distal shaft 76 can also be permanently optically and/or mechanically coupled to the camera unit 68. The camera unit 68 is arranged proximally with respect to the shaft 76. The camera unit 68 comprises an imaging sensor system 108, in the present case, for example, a white light sensor 110 and a near-IR sensor 112. Generally speaking, the imaging sensor system 108 can have one or more at least spatially resolving light sensors/image sensors, for example at least one CMOS sensor and/or at least one CCD sensor. The shaft 76 comprises optical elements (not shown) by means of which light can be guided to the camera unit 68 in order to be able to optically capture the object to be imaged. The shaft 76 further comprises at least one light path 114, for example defined by a light guide, such as an optical fiber, which leads to a distal portion 116 of the shaft 76 and by means of which the illumination light originating from the optical interface 16 of the illumination device 12 can be coupled out to the object to be imaged.

The camera unit 68 has different operating states, specifically for example at least one multispectral operating state and one fluorescence operating state and, in the present embodiments, additionally a white light operating state and possibly a hyperspectral operating state. The controller 66 automatically adapts the illumination mode of the illumination unit 18 to the current operating state of the camera unit 68. The controller 66 can set the image recording behavior of the camera unit 68. For example, the controller 66 can adjust the exposure time, sensitivity/amplification/gain and/or other operating parameters of the camera unit 68 or, in particular, its image capture sensor system 108 and, if applicable, its optics, thereby defining different operating states of the imaging unit 14. In the present case, the controller 66 triggers the illumination unit 18 synchronously with the camera.

The imaging unit 14 comprises a filter unit 46 with optical filters 48, 50 52. Three optical filters are shown as an example, but it shall be understood that a different number can be used. The filter unit 46 can be switched between a multispectral mode and a fluorescence mode. Moreover, the filter unit 46 can additionally be switchable to a white light mode and/or a hyperspectral mode. The optical filters 48, 50, 52 can optionally be introduced into an observation beam path 70 of the camera unit 68, thereby defining different observation modes. In the present case, they define the operating states of the camera unit 68.

A basic imaging mode can be associated with a plurality of optical filters 48, 50, 52. In particular for fluorescence imaging, a different suitable optical filter can be used depending on the luminous element 20, 22, 24, 26, 28 used for excitation. For example, in the present case the first luminous element 20 (red) is combined with an optical filter that transmits wavelengths greater than 730 nm but blocks shorter wavelengths. This makes it possible in particular to ensure that only fluorescent light and not the excitation light itself is detected. For example, said optical filter can absorb at least in the 600 nm to 730 nm range. Furthermore, in the present case, for example, the second luminous element 22 (dark red) is combined with a filter which absorbs in the 700 to 850 nm range or which only transmits significantly above 850 nm.

The user can select a specific filter 48, 50, 52 and thereby directly selects an associated observation mode or operating state of the camera unit 68. For this purpose, the camera unit 68 has a filter sensor 72 which can automatically detect an optical filter currently introduced into the observation beam path 70. The user can thus manually introduce a selected filter 48, 50, 52 into the observation beam path 70. In the example shown, the optical filters 48, 50, 52 are mounted on a filter carrier 118. Said filter carrier 118 can be moved into different positions, whereby one of the optical filters 48, 50, 52 can be selected in each case. The filter sensor 72 then detects the currently selected optical filter 48, 50, 52. The controller can then determine the current operating state of the camera unit 68 and thus of the imaging unit 14 based on a sensor signal from the filter sensor 72 and automatically adapt the illumination mode of the illumination unit 18 accordingly. The user thus puts the entire imaging device 10 into the desired mode by a simple user action, such as a manual selection of an optical filter 48, 50, 52. A user can basically combine different filters with different illumination modes to create different types of contrast.

In the illustrated case, the imaging unit 14 and in particular the shaft 76 comprises a broadband transmitting optics 77, which can be used uniformly in the different illumination modes. In the present case, the broadband optics 77 is designed for a spectral range of at least 400 nm to 1000 nm. It can be used uniformly for different illumination and/or observation spectral ranges.

In some embodiments, the imaging unit 14 can be configured as a stereoendoscope that comprises a stereoscopic ocular having two sides. Different optical filters can be connected upstream of these sides independently of one another, allowing different contrast images to be superimposed on one another.

In the following, in the context of further embodiments and modifications, the same reference signs as above will be used for identical or similar components. As regards their description, reference is generally made to the above embodiments, whereas primarily the differences between the embodiments will be explained below. Also, reference signs have been partially omitted in the following figures for reasons of clarity.

FIG. 5 shows a schematic representation of another embodiment of the imaging device 10. The imaging device 10 comprises an illumination device 12 having an optical interface 16, and an illumination unit 18 as well as an imaging unit 14 which is connected to the optical interface 16. The imaging unit 14 comprises a camera unit 68 having an automated filter unit 210. The automated filter unit 210 comprises a plurality of optical filters 48, 50, 52, which can be automatically introduced into an observation beam path 70 of the camera unit 68 in accordance with an observation mode specified by a user.

The automated filter unit 210 comprises a filter drive 212 which is configured to automatically move the optical filters 48, 50, 52 into or out of the observation beam path 70. The optical filters 48, 50, 52 can be mounted on a filter carrier 118 which is connected to the filter drive 212. The filter drive 212 can be configured to move, for example to shift and/or rotate and/or pivot, the filter carrier 118.

The imaging unit 14 has a user interface 214 by means of which the user can adjust a desired observation mode. For example, a desired position of the filter carrier 118 can be specified by means of the user interface 214.

The imaging unit 14 further has a controller 66. The controller 66 is coupled to the filter drive 212 and the user interface 214. The controller 66 is configured, in particular, to process a user specification of an observation mode and to control both the filter unit 210 and the illumination unit 18 in accordance with said user specification. The controller 66 can thus adjust an operating state of the imaging unit 14 and an illumination mode of the illumination unit 18 matched thereto in accordance with an observation mode selected by the user.

FIG. 6 shows a schematic representation of yet another embodiment of the imaging device 10. The imaging device 10 comprises an illumination device 12 having an optical interface 16, and an illumination unit 18 as well as an imaging unit 14 which is connected to the optical interface 16. The imaging unit 14 comprises a proximal base unit 310. The proximal base unit 310 is connected to the optical interface 16 of the illumination device 12. Illumination light generated by the illumination device 12 can thus be supplied to the proximal base unit 310. The imaging unit 14 further comprises a controller 66, which in some embodiments may be integrated in the base unit 310.

Optionally, different exchangeable shafts 312, 314 can be optically and electronically coupled to the proximal base unit 310. The base unit 310 has an interface 316 for coupling different exchangeable shafts 312, 314. Said interface 316 supplies the illumination light coming from the illumination device 12 to a coupled exchangeable shaft 312, 314. The interface 316 is further configured to electrically supply a coupled exchangeable shaft 312, 314 and/or to electronically connect it to the controller 66 of the imaging unit 14.

The exchangeable shafts 312, 314 each have an integrated camera 318, 320 and integrated optical filters 322, 324. The integrated cameras 318, 320 are configured as Tipcams. In the present case, the integrated camera 318 of a first exchangeable shaft 312 is configured for multispectral imaging. Furthermore, the integrated camera 310 of a second exchangeable shaft 314 is configured for fluorescence imaging. The optional optical filters 322, 324 can be adapted thereto.

In other embodiments, it is also possible to use exchangeable shafts that only include optical filters but no integrated camera. In that case, they can be couplable to a proximal camera unit. In some cases, the proximal camera unit can then be designed without an additional filter unit. The selection of a specific optical filter or a specific observation mode can be made by choosing a suitably equipped exchangeable shaft.

The controller 66 is configured to detect a coupled exchangeable shaft 312, 314. This can be done software-based, mechanically and/or through sensor detection. Depending on the detected exchangeable shaft 312, 314, the controller 66 can then determine in which operating state or in which observation mode the imaging unit 14 should be operated. The control unit 66 is also configured to adjust an illumination mode of the illumination unit 18. The control unit 66 is thus configured to adjust an illumination mode of the illumination unit 18 on the basis of the observation mode defined by a currently coupled exchangeable shaft 312, 314.

In the present case, the exchangeable shafts 312, 314 and the imaging device 10 are part of a medical imaging system 316. The medical imaging system 316 allows a user to select a suitable exchangeable shaft 312, 314, couple it to the base unit 310, and thereby define a mode for the entire imaging device 10. By simply changing the exchangeable shaft 312, 314, it is thus achieved that the illumination device 18 is automatically adapted to the image capture mode to be used.

FIG. 7 shows a schematic perspective representation of another embodiment of an imaging device 10′. The reference signs of this embodiment are provided with inverted commas for differentiation purposes. In this embodiment, the imaging device 10′ is designed as an exoscopic imaging device. It comprises an illumination device 12′ and an imaging unit 14′. Its basic functionality corresponds to that described above, but in this embodiment the imaging unit 14′ is configured as an exoscope.

Aspects of the above description can also be summarized or described as follows. FIG. 8 shows a schematic flow chart of a method for generating illumination light for an imaging unit 14 by means of an illumination device 12. The sequence of the method is also clear from the above explanations. The illumination device 12 comprises an optical interface 16 for the optical connection of an imaging unit 14, and an illumination unit 18 that is designed to provide illumination light to the optical interface 16, wherein the illumination unit 18 comprises a plurality of luminous elements 20, 22, 24, 26, 28 which are optionally activatable independently of one another and which are configured to emit light according to different emission spectra in order to provide the illumination light.

The method comprises a step S11 of at least temporarily activating a first group of the luminous elements 20, 22, 24, 26, 28 in order to provide illumination light for multispectral imaging. The method further comprises a step S12 of at least temporarily activating a second group of the luminous elements 20, 22, 24, 26, 28 in order to provide illumination light for fluorescence imaging. One of the luminous elements 20, 22, 24, 26, 28 is activated at least temporarily both when the first group of luminous elements 20, 22, 24, 26, 28 is activated at least temporarily and when the second group of luminous elements 20, 22, 24, 26, 28 is activated at least temporarily.

FIG. 9 shows a schematic flow chart of a method for operating an imaging device 10. The sequence of the method is also clear from the above explanations. An imaging device 10 having an imaging unit 14 is provided in a step S21. Illumination light is provided to the imaging unit 14 in a step S22. The provision of illumination light to the imaging unit 14 is carried out according to a method as described with reference to FIG. 8.

FIG. 10 shows a schematic flow chart of a method for operating an imaging device 10. The sequence of the method is also clear from the above explanations. The method comprises a step S31 of providing an illumination device 12 for providing illumination light for an imaging unit 14. The imaging unit 14 comprises an optical interface 16 for the optical connection of an imaging unit 14, and an illumination unit 18 that is designed to provide illumination light to the optical interface 16. The illumination unit 18 is multimodal and can be operated in a plurality of different illumination modes. The method further comprises a step S32 of providing an imaging unit 14 that can be connected to the optical interface 16 of the illumination device 12. The method moreover comprises a step S33 of automatically matching an operating state of the imaging unit 14 and an illumination mode of the illumination unit 18.

The following describes an aspect concerning the generation of displays from fluorescence images and anatomy images. FIG. 11 shows a schematic representation of a medical imaging device 410 according to this aspect. The medical imaging device 410 can basically be constructed and/or designed like the imaging device 10 described above or also like the above imaging device 10′. In particular, reference is made to the above description regarding the functionality of the components and details of the design of the imaging device 410. In order to explain the present aspect, it is expedient to describe the technical facts with reference to FIG. 11, which is to be understood purely schematically, and the other figures.

The imaging device 410 is an endoscope device in the specific example, but can also be an exoscope device and/or a microscope device. The imaging device 410 comprises an illumination unit 412 having at least one light source 414. The illumination unit 412 can, for example, be designed as described above with reference to the illumination device 12. For the following description, it is assumed that the illumination unit 412 is designed in this way. However, this is to be understood as purely exemplary. Basically, the illumination unit 412 is configured to provide illumination light 432, 438, by means of which an object 418 to be imaged can be illuminated. This will be discussed in more detail below.

The imaging device 410 further comprises an image capture unit 420 with suitable image capture sensors 422. The image capture sensor system 422 is configured here to be able to record images in both the visible range and the near-infrared range. For example, the image capture sensor 422 is sensitive at least in a range between 450 nm and 950 nm, in some embodiments in a range between 400 nm and 1000 nm.

The object 418 to be imaged is, for example, an anatomical structure, such as in a cavity of a patient. The anatomical structure comprises a range marked with a fluorescent dye 454. Using the imaging device 410, a user can view the anatomical structure as well as the marked region 454 and use the fluorescence of the marked region 454 to distinguish it from the surrounding tissue. As an application by way of example, reference is made to a free preparation of the marked region 454, whereby a person skilled in the art is also familiar with other applications of fluorescent labeling of tissue.

In the present case, the marked range 454 is marked with a fluorescent dye which absorbs light in the visible range and also fluoresces at least partly or exclusively in the visible range. An absorption spectrum and an emission spectrum are shown by way of example in the upper region of FIG. 12 as a solid or dash-dotted line in a diagram in which an intensity is plotted against the wavelength. To excite the dye, light is irradiated with a wavelength that lies in the absorption range of the dye. This is shown in FIG. 12 by a dashed line, with the corresponding wavelength marked as a point on the x-axis. This is the illumination light 432, which is shown as an arrow in FIG. 11.

The dye, for example, is fluorescein. This dye can expediently be excited at a wavelength of 430 nm or 460 nm. In particular, this can be done by means of the third luminous element 24 described above. Another example of the dye is Cy5. This dye can expediently be excited at a wavelength of 660 nm. In particular, this can be done by means of the first luminous element 20 described above.

In order to perform fluorescence imaging, the image capture unit 420 comprises an observation filter 424. Said filter is mounted in front of the image capture sensor system 422 and/or integrated into it and ensures that the illumination light 432 is not remitted to the image capture sensor system 422 or at least not to individual sensors thereof, but exclusively or at least primarily light 434 which is emitted by the object 418 due to the fluorescence excitation of the dye.

In the lower region of FIG. 12, a suitable transmission spectrum of the observation filter 424 is shown, which allows such fluorescence imaging. The observation filter 424 is configured to block light in a first spectral range 426 and to transmit light in a second spectral range 428 which differs from the first spectral range 426. The observation filter 420 is suitable to be selected depending on the dye used or the absorption spectrum and emission spectrum. As described above, the imaging device 410 may comprise a plurality of optical filters that can be selectively used as observation filters. As a result, fluorescence imaging for different fluorescent dyes can be performed using the imaging device 410.

The observation filter 424 with the transmission shown in FIG. 12 is a high-pass edge filter. In the region of the edge, the transmission behavior of the observation filter 424 changes abruptly, although the corresponding edge may still have a certain width. The region of the edge may not fall into either of the two spectral ranges 426, 428.

Depending on the position, width and shape of the absorption peak of the dye in question, a two-edge observation filter may be used instead of a single-edge observation filter 424. This is illustrated by way of example in FIG. 13. The first spectral range 426′ lies here between two sub-ranges of the second spectral range 428′. Light is therefore transmitted both above and below the first spectral range 426′. In this case, the observation filter only masks out a sub-range of the observable spectral range that lies in the visible range.

The image capture unit 420 is further configured to operate in a white light mode or an anatomy mode in which the object 418 is illuminated by means of illumination light 438 which lies at least partly within the second spectral range 428 and in which light 440 remitted by the object 418 is detected. As shown schematically in FIG. 11, a portion of the remitted light 440 is blocked by the observation filter 424 and the portion that lies outside the first spectral range 426 or in the second spectral range 428 is passed through to the image capture sensor 422. If the first spectral range 426 is selected to be in the visible range due to the dye used, a complete white light image cannot be recorded even if white light is irradiated. This can be done, for example, by means of a white light luminous element, as described above.

Reference is also made below to FIG. 14. The image capture unit 420 is configured to record fluorescence images 430 of the object to be imaged through the observation filter 424 in the manner described. Furthermore, the image capture unit 420 is configured to record anatomy images 436 of the object 418 to be imaged in the manner described. In the present case, the anatomy images 436 are images in which broadband illumination light is irradiated and remitted light is detected. FIG. 14 shows the case where several anatomy images 436 and several fluorescence images 430 are recorded or obtained from corresponding image data over time. Specifically, these are either recorded sequentially by alternately recording one or more anatomy images 436 and then one or more fluorescence images 430 (or vice versa) according to a specific logic, or by recording anatomy images 436 and fluorescence images 430 simultaneously. This depends on the design and operating mode of the image capture sensor system 422.

The anatomy images 436 are not true white light images because they were recorded through the observation filter 424. Rather, they are obtained by processing individual images and/or color channels according to one or more of the principles described below. The imaging device 410 further comprises a display generation unit 442 which is configured to generate a display 444 from the fluorescence images 430 and the anatomy images 436. In the present case, this comprises a combination display and/or an overlay display based on a combination or overlay of one or more anatomy images 436 and one or more fluorescence images 430.

The display generation unit 442 is configured to associate recorded fluorescence images 430 and recorded anatomy images 436 with each other. This can be done both in the case of sequential image recording and in the case of simultaneous image recording. In the case presented, it is a matter of temporal assignment. The assignment is based on the information about when the images were recorded, so that the display 444 contains temporally related anatomy information and fluorescence information. For example, a user is provided with a moving image in real time.

As shown schematically in FIG. 11, the imaging device 410 comprises a visual display 452 via which the generated display 444 can be displayed to a user. This design is to be understood purely as an example. In particular, in other embodiments, a visual display may be connectable to the imaging device 410 only via an interface but may not necessarily be part of the same.

As mentioned, even with broadband illumination, image information is not available over the entire visible range when recording the anatomy images 436 due to the observation filter 424. If regular white light imaging were performed, the white light image in question would be distorted in color or might not correctly depict specific anatomical structures.

It is therefore intended that, in addition to image information in the visible range, image information from the near-infrared range is also used to generate the anatomy image 436. This allows information that may not be available due to the observation filter 424 to be provided elsewhere.

Purely by way of example, this is shown in FIG. 15 for four color channels of an image, as it would result from illumination with white light and near-infrared light. For the purposes of explanation, it should be assumed that specific anatomical structures or other image information are only recognizable in the blue (B), green (G) or red (R) color channel and/or individual image. If these color channels and/or individual images B, G, R were completely combined, a correct white light image of the object 418 would be created. However, since the imaging takes place through the observation filter 424, the image information from the blue color channel and/or blue individual image, for example, is missing. If only the green and red color channels and/or the green and red individual images were combined, an incomplete image would result. The present aspect, however, provides for the addition of a near-infrared color channel (NIR) and/or a near-infrared individual image. This may make it possible to identify details of the anatomical structure that would be visible in the unavailable spectral range (here, by way of example, in the blue). If this additional image information is now combined with the available color channels and/or individual images, an anatomy image 436 can be obtained that correctly represents the anatomy, although it is not a true white light image.

Anatomy images 436 obtained in this way are then combined with fluorescence images 34 in the manner described to generate the display 444 which contains information regarding both anatomy of the object 418 and fluorescence of the marked region 454.

Below, various possibilities are described as to how anatomy images 436 can be obtained for different designs of the image capture sensor system 422 and/or different approaches to the utilization of available information. It is assumed in each case that a multimodal illumination unit is used. For example, and in accordance with the embodiment described above, this has a white light image luminous element and a blue luminous element which emits, for example, at 430 nm or 460 nm, a red luminous element which emits, for example, at 660 nm, a far-red luminous element which emits, for example, at 765 nm or 770 nm, and a near-infrared luminous element which emits, for example, at 940 nm. As already mentioned, however, this is primarily for the sake of comprehensibility, and other illumination units can also be used.

FIG. 16 illustrates a first example in which the image capture sensor system 422 comprises a two-chip image sensor. Imaging light is distributed across two sensors, as described above by way of example. A first image sensor (VIS) operates in the visible range, for example at wavelengths up to approximately 700 nm, and a second image sensor (NIR) operates in the near-infrared range, for example at wavelengths from about 700 nm. In this example, fluorescein is used as the dye. Said dye is excited by the blue luminous element. Due to the observation filter 424, blue light cannot be detected. In addition to the illumination for the excitation of the dye, illumination is provided, for example, with the far-red luminous element and/or with the near-infrared luminous element.

Fluorescence images 430 are obtained using the VIS image sensor. Anatomy images 436 are obtained using the NIR image sensor. The anatomy image 436 is output as a grayscale image, so that the detection in the near-infrared range, which is not perceptible to the user, does not pose an obstacle. The display generation unit 442 thus generates a display 444 that superimposes a grayscale anatomy image 436 and a fluorescence image 34 on each other. Both the fluorescence images 430 and the anatomy images 436 can be recorded in real time, for example at least 24 fps, at least 30 fps or even at least 60 fps. In addition, since different image sensors are used to capture the fluorescence image 430 and the anatomy image 436, they can be captured simultaneously. Sequential recording is not required.

FIG. 17 illustrates a second example in which the image capture sensor system 422 also comprises a 2-chip image sensor. As before, a fluorescence image 430 is recorded using the VIS image sensor. However, unlike the first example, anatomy images 436 and fluorescence images 430 are recorded sequentially. This makes it possible to use not only the NIR image sensor but also the VIS image sensor for the anatomy image 436.

The VIS image sensor provides three color channels 436B, 436G, 436R, which can basically be used for the anatomy image 436. Due to the observation filter 424, the blue color channel 436B is hardly or not at all usable, since little to no light remitted by the object 418 passes through the observation filter 424 in the corresponding spectral range. The blue color channel 436B is therefore disregarded here. In contrast, both the green color channel 436G and the red color channel 436R of the VIS image sensor image data as well as a near-infrared individual image 436NIR of the NIR image sensor are used to obtain the anatomy image 436. The anatomy image 36 is reconstructed by replacing the blue channel with the near-infrared channel or the near-infrared individual image. This allows a colored anatomy image to be created. However, due to the sequential recording of anatomy images 436 and fluorescence images 430, the frame rate is halved in this example.

In some applications, a lower refresh rate may be acceptable for fluorescence information than for anatomy information. For example, assuming an available frame rate of 60 fps, several anatomy images, such as two or three or five, can always be recorded one after the other before a fluorescence image is acquired. The display generation unit then assigns the most recently recorded fluorescence image to each of the several anatomy images. For example, this approach can record 50 anatomy images and 10 fluorescence images per second. In this example, the actual refresh rate of the fluorescence information is only 10 fps, which may not be perceptible to the user due to the higher refresh rate of the anatomy information.

FIG. 18 illustrates a third example. In this example, the image capture sensor system 422 comprises a one-chip (OC) image sensor. It uses a red, a green and a blue channel and is also able to detect near-infrared light on all three channels. In the example, the dye Cy5 is excited. This is done, for example, by means of a red luminous element.

Anatomy images 436 and fluorescence images 430 are recorded sequentially. For the fluorescence image 430, the image data of the image sensor can be used unchanged.

In this case, the observation filter 424 blocks out a first spectral range 426 which extends from approximately 600 nm to approximately 700 nm. The second spectral range 428 lies both above and below the first spectral range 426 (see the display by way of example in FIG. 13). Light in the first spectral range 28 is not detected due to the observation filter 424. However, when an anatomy image 436 is recorded, the red channel still provides a signal because near-infrared light is captured by all three channels of the image sensor. The anatomy image 436 is calculated in this example by combining the three channels in an appropriate manner. An anatomy image 436 with the color channels R′G′B′ is obtained, for example, by using the red channel of the image sensor as the red channel of the anatomy image 436, i.e., R′=R, by using a difference between the green and red channels of the image sensor as the green channel G′, i.e., G′=GR, and by using a difference between the blue and red channels of the image sensor as the blue channel of the anatomy image 436, i.e., B′=BR.

It is understood that any other combinations, in particular linear combinations, can be used. These may also be weighted depending on the response of the image sensor in question and/or depending on the observation filter 424 used. As a further example, in the case of excitation of fluorescein, where blue light would be blocked, the color channels of an anatomy image 436 could be calculated as follows: R′=R−B; G′=G−B; B′=B.

FIG. 19 illustrates a fourth example. In this example, the image capture sensor system 422 comprises a four-chip image sensor with separate image sensors for a blue channel (B), a green channel (G), a red channel (R), and a near-infrared channel (NIR). Light coming from object 418 is divided between these four sensors. In this example, an illumination unit may also be used which, instead of a white light luminous element, comprises a combination of three or more luminous elements by means of which light is mixed in such a way that it can be used instead of white light. For example, a combination of red, green and blue luminous elements can be used. Although they only emit in specific and optionally very narrow-band wavelength ranges, they can provide illumination for recording a red, green and blue image, so that an image can be obtained that is perceived by a user as a white light image. In this case, for example, the single-colored luminous elements mentioned above are present and, in addition, the combination of colored luminous elements mentioned above, which are used instead of the white light luminous element.

In principle, this configuration can be operated analogously to the first and second examples described above. Then, for example, the observation filter 424 is arranged in front of the four-chip image sensor. In the following, another variant will be explained which can be implemented using the system according to the fourth example.

Fluorescein, for example, is again used as a dye. To stimulate the dye, light is shined through a blue luminous element. Remitted light is in the green range and is detected by the green image sensor. A filter integrated into the green image sensor and/or arranged in front of the image sensor, which makes it selective for green light, serves as an observation filter 424. The image of the green image sensor can thus be used as a fluorescence image 430, since the blue excitation light does not fall on the green image sensor.

In order to obtain an additional anatomy image, illumination is provided by a near-infrared luminous element, for example. The object thus remits light in both the blue and near-infrared range. In these regions, there is no fluorescence of the dye used. The dye fluorescein emits predominantly in the green range, but its emission spectrum also extends into the red with low intensity. The green and red image sensors are therefore not used for the anatomy image 436. The anatomy image 436 is thus obtained by combining a blue individual image 436B recorded by the blue image sensor and a near-infrared individual image 436NIR recorded by the near-infrared image sensor.

It is understood that an analogous procedure can be followed if another dye is used which, for example, can be excited with green light and emits red light.

FIG. 20 shows a schematic flow diagram of a method for medical imaging. The sequence of the method also follows from the above explanations. In a step S41, illumination light 438 is provided for illuminating an object 418 to be imaged. In a step S42, fluorescence images 430 of the object 480 to be imaged are recorded through an observation filter 424 which is configured to block light in a first spectral range 426 which comprises visible light and to transmit light in a second spectral range 428 which differs from the first spectral range 426, wherein when recording the fluorescence images 430, illumination light 432, the spectrum of which lies at least partly within the first spectral range 426, is used as excitation light, and light 434 which is emitted from the object 418 to be imaged, the spectrum of which lies at least partly within the second spectral range 428, is detected. Furthermore, the method comprises a step S43 in which anatomy images 436 of the object 418 to be imaged are recorded, wherein during the recording of the anatomy images 436, illumination light 438, the spectrum of which lies at least partly within the second spectral range 428, is used and light 440 which is remitted from the object 418 to be imaged, the spectrum of which lies at least partly within the second spectral range 428, is detected.

FIG. 21 shows a schematic display of a computer program product 448 with a computer-readable medium 450. The computer-readable medium has stored therein program code which, when executed in a processor, is designed to carry out one and/or all of the described methods.

### LIST OF REFERENCE SIGNS

10 Imaging device
12 Illumination device
14 Imaging unit
16 Optical interface
18 Illumination unit
20 Luminous element
22 Luminous element
24 Luminous element
26 Luminous element
28 Luminous element
30 Beam splitter
32 Beam splitter
34 Input side
36 Input side
37 Input side
38 Input side
40 Input side
41 Input side
42 Output side
44 Output side
46 Filter unit
48 Filter
50 Filter
52 Filter
54 Optical path
56 Light-emitting surface
58 Light-emitting surface
60 Light-emitting surface
62 Light-emitting surface
64 Light-emitting surface
66 Controller
68 Camera unit
70 Observation beam path
72 Filter sensor
74 Display unit
76 Shaft
77 Optics
78 Lens
80 Lens
82 Lens
84 Lens
86 Lens
88 Lens
90 Beam splitter element
92 Beam splitter element
94 Beam splitter element
96 Beam splitter element
98 Transmission spectrum
100 Transmission spectrum
102 Transmission spectrum
104 Transmission spectrum
106 Light guide
108 Imaging sensor system
110 White light sensor
112 Near-IR sensor
114 Light path
116 Distal portion
210 Filter unit
212 Filter drive
214 User interface
310 Base unit
312 Exchangeable shaft
314 Exchangeable shaft
316 Imaging system
318 Camera
320 Camera
322 Filter
324 Filter
410 Imaging device
412 Illumination unit
414 Light source
416 Illumination light
418 Object
420 Image capture unit
422 Image capture sensor system
424 Observation filters
426 First spectral range
428 Second spectral range
430 Fluorescence image
432 Illumination light
434 Light
436 Anatomy image
438 Illumination light
440 Light
442 Display generation unit
444 Display
448 Computer program product
450 Computer-readable medium
452 Display

1-16. (canceled)

17. A medical imaging device comprising:
an illumination unit having at least one light source that is configured to provide illumination light to illuminate an object to be imaged; and
an image capture unit that comprises at least one image capture sensor system and at least one observation filter,
wherein the observation filter is configured to block light in a first spectral range that comprises visible light, and to transmit light in a second spectral range that differs from the first spectral range;
wherein the image capture unit is configured to record, through the observation filter, fluorescence images of the object to be imaged, during the recording of the fluorescence images, illumination light, the spectrum of which lies at least partly within the first spectral range, is used as excitation light and, during the recording of the fluorescence images, light which is emitted from the object to be imaged, the spectrum of which lies at least partly within the second spectral range, is detected by the image capture sensor system, and
wherein the image capture unit is configured to record anatomy images of the object to be imaged, during the recording of the anatomy images, illumination light, the spectrum of which lies at least partly within the second spectral range, is used and, during the recording of the anatomy images, light which is remitted from the object to be imaged, the spectrum of which lies at least partly within the second spectral range, is detected by the image capture sensor system; and
a display generation unit which is configured to generate at least one display from the fluorescence images and the anatomy images.

18. The medical imaging device claim 17, wherein the image capture unit is configured to record the anatomy images of the object to be imaged through the observation filter.

19. The medical imaging device of claim 18, wherein a smallest wavelength of the first spectral range is greater than 430 nm and in particular greater than 500 nm.

20. The medical imaging device of claim 1, wherein the at least one display comprises a combination display that is based on a combination of fluorescence images and anatomy images.

21. The medical imaging device of claim 20, wherein the combination display is an overlay display.

22. The medical imaging device of claim 1, wherein the image capture unit is configured to record the fluorescence images and the anatomy images in real time, and wherein the display generation unit is configured to generate the at least one display in real time.

23. The medical imaging device of claim 1, wherein the image capture unit is configured to record the fluorescence images and the anatomy images sequentially, and wherein the display generation unit is configured to assign sequentially recorded fluorescence images and anatomy images to one another and to generate the at least one display based on assigned fluorescence images and anatomy images.

24. The medical imaging device of claim 1,
wherein the image capture sensor system comprises at least one first image capture sensor (VIS, NIR, OC, R, G, B) which is configured to capture image information for the fluorescence images, and
wherein the image capture sensor system comprises at least one second image capture sensor (VIS, NIR, OC, R, G, B) which is configured to capture image information for the anatomy images.

25. The medical imaging device of claim 1, wherein the image capture sensor system is configured to simultaneously record fluorescence images and anatomy images.

26. The medical imaging device of claim 1, wherein the display generation unit is configured to generate at least one anatomy image from a plurality of, in particular single-colored, individual images and/or from a plurality of color channels of anatomy images.

27. The medical imaging device of claim 1, wherein the anatomy image comprises at least two color channels, of which at least one lies in the visible range and at least one lies outside the visible range.

28. The medical imaging device of claim 27, wherein the at least one color channel outside the visible range is in the near infrared range.

29. The medical imaging device of claim 1, wherein the anatomy image corresponds to a white light image with multiple color channels, in which at least one color channel from the visible range is replaced by at least one color channel that lies outside the visible range.

30. The medical imaging device of claim 29, wherein the one color channel that lies outside the visible range lies in the near infrared range.

31. The medical imaging device of claim 1, wherein the illumination unit is multimodal and comprises a plurality of luminous elements which are selectively activatable independently of one another and which are configured to emit light according to different emission spectra in order to provide the illumination light.

32. The medical imaging device of claim 1, wherein the image capture unit is configured for multispectral and/or hyperspectral imaging.

33. A method for medical imaging, comprising:
providing illumination light to illuminate an object to be imaged;
recording fluorescence images of the object to be imaged through an observation filter that is configured to block light in a first spectral range that comprises visible light, and to transmit light in a second spectral range that differs from the first spectral range, wherein, during the recording of the fluorescence images, illumination light, the spectrum of which lies at least partly within the first spectral range, is used as excitation light, and light which is emitted from the object to be imaged, the spectrum of which lies at least partly within the second spectral range, is detected; and
recording anatomy images of the object to be imaged, wherein during the recording of the anatomy images, illumination light, the spectrum of which lies at least partly within the second spectral range, is used, and light which is remitted from the object to be imaged, the spectrum of which lies at least partly within the second spectral range, is detected.

34. A program code which, when executed in a processor, is configured to cause an illumination light to illuminate an object to be imaged;
record fluorescence images of the object to be imaged through an observation filter that is configured to block light in a first spectral range that comprises visible light, and to transmit light in a second spectral range that differs from the first spectral range, wherein, during the recording of the fluorescence images, illumination light, the spectrum of which lies at least partly within the first spectral range, is used as excitation light, and light which is emitted from the object to be imaged, the spectrum of which lies at least partly within the second spectral range, is detected; and
record anatomy images of the object to be imaged, wherein during the recording of the anatomy images, illumination light, the spectrum of which lies at least partly within the second spectral range, is used, and light which is remitted from the object to be imaged, the spectrum of which lies at least partly within the second spectral range, is detected.

35. The program code of claim 34 further comprising a computer-readable medium on which program code is stored.

36. The program code of claim 35, further comprising a computer program product that comprises the computer-readable medium.
